(19) United States
(12) Patent Application Publication
(10) Pub. No.: US 2002/0052581 A1
(43) Pub. Date: May 2, 2002

(54) Hollow needle

(76) Inventors: Ulrich J. Pfeiffer, Munchen (DE); Thomas Thalmeier, Munchsmunster (DE)

Correspondence Address: NIXON PEABODY, LLP, 8180 GREENSBORO DRIVE, SUITE 800, MCLEAN, VA, 22102, US

(21) Appl. No.: 09/803,982

(22) Filed: Mar. 13, 2001

(30) Foreign Application Priority Data
Oct. 31, 2000 (DE) 100 53 883.5

(51) Int. Cl. A61M 5/32

(52) U.S. Cl. 604/272

(57) ABSTRACT

The outer diameter of a hollow needle, comprising a ground surface at its tip, expands continuously from a region near the tip but behind the ground surface such that the outer diameter in the region located further from the tip is approximately double. The rotationally symmetrical transition region is conical and rounded off at each end. In use, the ground tip surface of the puncture needle cuts through the skin, the intermediate tissue and the wall of the blood vessel. The wider part of the hollow needle causes the puncture point to widen. Due to having a primary puncture of small diameter and then widening the puncture hole to the diameter required for introducing a correspondingly thick catheter, the traumatic injury to the blood vessel thereafter recedes relatively quickly back to the smaller diameter of the puncture. Accordingly, the time for healing a wound is limited to that required for a smaller hole.

### BACKGROUND OF THE INVENTION

1. Field of the Invention

The invention relates to a hollow needle for puncturing blood vessels and for introducing a wire, especially for the placement of catheters or the like.

2. Description of the Related Art

Hollow needles of this type, which are also referred to as puncture needles or cannulas, are employed, inter alia, for the application of a catheter by means of the so-called Seldinger technique. This process generally proceeds as follows: The blood vessel, into which the catheter is to be placed, is punctured with the hollow needle. By means of the hollow needle there is introduced a guide wire, which remains in the blood vessel while the hollow needle is then withdrawn. Before the catheter is threaded onto the guide wire and is introduced into the blood vessel thereby, it is usually necessary to widen the puncture point by employing a so-called dilator. A dilator is a sleeve of plastic material which extends conically towards one end and is guided to the puncture point by the guide wire in order to widen it out to match the outer diameter of the catheter.

### SUMMARY OF THE INVENTION

Based upon the background of the general requirements in the medical field, that every operation on the human body should be carried out as carefully as possible and with a low level of risk, that it should not be drawn out unduly and that the number of avoidable steps involved in an operation be reduced correspondingly, the object of the invention is to produce a device which, in use, will speed up and simplify the placement of the catheter in the blood vessels without dispensing with the advantages associated with the use of a conventional dilator.

In accordance with the present invention, this object is achieved by a hollow needle for puncturing blood vessels and for introducing a wire, especially for the placement of catheters or the like, whereby the needle has a first outer diameter in a first region near its tip, and has a second outer diameter in a second region which is located further from the tip than the first region, the second outer diameter being greater than the first outer diameter, preferably being approximately 1.2 to 3.0 times greater. Such a needle has the advantage that the puncturing and careful widening of the puncture point is done in one step. The advantages of a conventional dilator are thereby retained but without having to utilize such a dilator. By dispensing with the dilator, one less auxiliary means needs to be obtained, stored and sterilized, so that apart from the costs for the actual operation, the accompanying costs are also reduced.

Preferably, the needle has a ground surface in the vicinity of the tip. In one advantageous embodiment of the present invention, the first outer diameter is between approximately 0.4 and 1.5 mm. Preferably, the cross section of the hollow needle expands continuously in a transition region from the first outer diameter to the second outer diameter. It is expedient if the transition region has a conical shape, whereby the opening or aperture angle between the longitudinal axis of the hollow needle and an envelope line lying in a common plane in the conical transition region preferably lies between approximately 10° and 45°. The transition region advantageously begins behind the ground surface. It is expedient if the transition between the first and the second outer diameter is rounded off.

In a preferred embodiment of the present invention, the regions having the first and second outer diameters and the transition region are formed in one piece. The reduced expenditure in comparison with the use of a dilator as an additional help means in particularly noticeable hereby.

### BRIEF DESCRIPTION OF THE DRAWINGS

An embodiment of the present invention will be explained hereinafter with the help of the schematic drawings which are not drawn to scale and in which:

FIG. 1 shows a schematic view of the front part of a hollow needle in accordance with the invention; and

FIG. 2 shows a longitudinal section through the front part of a hollow needle in accordance with the invention illustrated in FIG. 1, whereby the needle is rotated about its longitudinal axis by 90°.

### DESCRIPTION OF THE PREFERRED EMBODIMENT

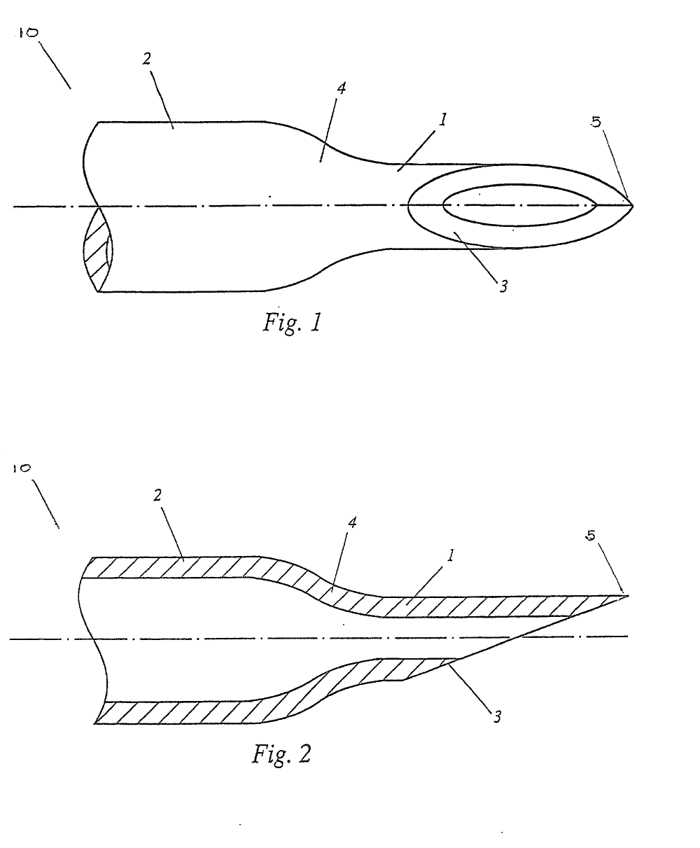

And now to the drawings, in which FIGS. 1 and 2 show a hollow needle having a body 10 having a first region 1, a second region 2, and a ground surface 3 at a tip end 5 located at a distal end of the body 10. Between the first region 1 and the second region 2 of the body 10 is a rotationally symmetrical transition region 4 having a conical shape that is rounded off at each end. Preferably, the transition region 4 has an opening angle lying between approximately 10° and 45°. The outer diameter of the body expands continuously from the first region 1 adjacent the tip but behind the ground surface 3 such that the outer diameter at the second region 2 located further longitudinally from the tip is approximately double that in region 1. Preferably, the outer diameter at the first region 1 is between approximately 0.4 mm and 1.5 mm and the ratio of the outer diameter at the second region 2 to the outer diameter at the first region 1 is between approximately 1.2 and 3.0. As a result, the body has a cross section expanding continuously in the transition region 4 from the outer diameter at the first region 1 to the outer diameter at the second region 2.

In use, the ground surface 3 of the hollow needle 10 or cannula cuts through the skin, the intermediate tissue and the wall of the blood vessel while the second region 2 of the hollow needle 10 causes the puncture point to widen. Consequently, it is possible, in a single operational step, to gain those advantages that were only obtained with the additional employment of a dilator when using conventional puncture needles. Due to having a primary puncture of small diameter and then widening the puncture hole to the diameter required for introducing a correspondingly thick catheter, the traumatic injury to the blood vessel thereafter recedes relatively quickly back to the smaller diameter of the puncture. This provides an advantage in contrast to a conventional needle having a uniform thickness and uniform outer diameter, since the time for healing the wound is limited to that required for a smaller hole. The healing process is thereby reduced and the patient is cared for as fully as possible.

It will be understood that the above description of the present invention is susceptible to various modification, changes and adaptations.

I claim:

1. A hollow needle for puncturing blood vessels and for introducing a wire for the placement of catheters, said hollow needle comprising:
a body having a tip end for insertion into a blood vessel, said tip located at a distal end of said body, a first outer diameter located in a first region adjacent said tip end, and a second outer diameter located in a second region located at a longitudinal distance further away of said distal tip end than said first region,
wherein said second outer has a diameter greater than the diameter of said first outer diameter.

2. A hollow needle in accordance with claim 1, wherein said body further includes a ground surface adjacent said distal tip.

3. A hollow needle in accordance with claim 1, wherein a ratio of said second outer diameter to said first outer diameter is between approximately 1.2 and 3.0.

4. A hollow needle in accordance with claim 1, wherein said first outer diameter is between approximately 0.4 mm and 1.5 mm.

5. A hollow needle in accordance with claim 1, wherein said body has a cross section expanding continuously in a transition region from said first outer diameter to said second outer diameter.

6. A hollow needle in accordance with claim 5, wherein said body has a conical shape in said transition region.

7. A hollow needle in accordance with claim 6, wherein said transition region has an opening angle lying between approximately 10° and 45°.

8. A hollow needle in accordance with claim 5, wherein said body further includes a ground surface located adjacent to said tip, said transition region located aft of said ground surface.

9. A hollow needle in accordance with claim 5, wherein said transition region between said first and the second outer diameters is rounded off.

10. A hollow needle in accordance with claim 1, wherein said first region, said second region and said transition region are formed as one piece.
